(19) United States
(12) Patent Application Publication
(10) Pub. No.: US 2004/0101955 A1
(43) Pub. Date: May 27, 2004

(54) Roller bottle

(76) Inventor: Kenneth W. Whitley, Raleigh, NC (US)

Correspondence Address: BECTON, DICKINSON AND COMPANY, 1 BECTON DRIVE, FRANKLIN LAKES, NJ, 07417-1880, US

(21) Appl. No.: 10/666,157

(22) Filed: Sep. 19, 2003

Related U.S. Application Data
(60) Provisional application No. 60/412,203, filed on Sep. 20, 2002.

(51) Int. Cl. C12M 1/24

(52) U.S. Cl. 435/304.1

(57) ABSTRACT

A container for cell growth culturing is provided that includes at its bottom end a neck-receiving recess having a planar wall with projecting ribs extending therefrom to space the planar wall from the neck of an adjacently stacked similar container. The ribs of the inventive container allow gas exchange with the inside of an adjacently-stacked similar container provided with a gas-permeable membrane.

### BACKGROUND OF THE INVENTION

1. Field of the Invention

This invention relates to a container for cell culture production and more particularly to a roller bottle having a recessed portion at its bottom end for accommodating an adjacently stacked roller bottle in a manner which allows gases to enter a gas-permeable cap of the stacked bottles.

2. Description of Related Art

One type of container commonly used in the laboratory for culturing of cells is known as a “roller bottle”. Roller bottles are generally cylindrically shaped and are adapted to rotate about their axes. The internal surfaces of such roller bottles are for providing active surfaces for cells. A liquid growth medium is introduced into the roller bottle. The rotating movement of the bottle keeps the internal surfaces wetted with a liquid medium, thereby encouraging the growth of cells. Rotating rollers of an appropriate apparatus are employed to rotate these roller bottles. The roller bottles are typically arranged on the rollers of the apparatus in end-to-end, stacked relationship, with one end of one bottle being abutted against the opposite end of an adjacent bottle.

It is desirable to grow large amounts of cells, mostly for cell by-products, such as pharmaceutical substances that are secreted by cells. The standard roller bottles have been successful in increasing the yield of cell growth insofar as the entire inside peripheral surface area can be utilized for cell culturing. In general, cell yields can be increased by maintaining ideal conditions for cell growth.

In typical culture systems, pH is maintained near physiologic levels by utilizing a buffering system in the tissue culture fluid, in conjunction with an incubator in which carbon dioxide (CO₂) is infused at a rate sufficient to maintain a concentration in the incubator atmosphere of approximately 5-7 volume percent. The CO₂ reacts with water to form a weak acid and a carbonic acid, which in turn inter-reacts with the buffering system to maintain the pH near physiologic levels. Entry of CO₂ from the incubator into the tissue culture vessel is generally achieved by utilizing a closure on the vessel such as, a loose fitting cap, a stopper or a cap with a permeable membrane. Equilibration in the vessel is maintained by allowing gas exchange with the inside of the vessel and the atmosphere of the incubator while preserving sterility and preventing liquid leakage.

One of the problems associated with roller bottles provided with caps including gas-permeable membranes has been that the cap can seal against the bottom of an adjacently stacked bottle when the two are stacked end-to-end. This can result in a gas-tight seal which prevents gases from flowing in and out of the permeable membrane, discouraging cell growth within the bottle.

A need exists, therefore, for an improved roller bottle having a means for allowing gases to exchange with the inside of a similar roller bottle with which it is adjacently stacked. By preventing a gas-tight seal from forming between the two stacked roller bottles, the cell cultures would be prevented from exposure to undesirable changes in the pH of the system.

### SUMMARY OF THE INVENTION

The present invention provides a container for cell growth culturing, such as a roller bottle. The container includes an elongate cylindrical wall having a closed bottom end and an opposed projecting neck portion defining a liquid opening. The closed bottom end includes an inwardly directed recessed portion including a planar surface. The recessed portion accommodates a neck portion of an adjacently stacked similar container. The planar surface includes a plurality of ribs extending therefrom so as to space the neck portion of the similar container from the planar wall.

The invention further provides a container assembly including the container just described; and a venting cap including a gas-permeable membrane for closing the liquid opening.

The invention solves a need in the art by providing ribs at the bottom of the container which prevent the bottom of the bottle from sealing against the top of a similar bottle when the two are stacked end-to-end.

### BRIEF DESCRIPTION OF THE DRAWINGS

FIG. 1 is a longitudinal cross-section of a roller bottle of the invention.

FIG. 1A is a partial sectional view of the neck portion of the roller bottle of FIG. 1.

FIG. 1B is a partial sectional view of a cap for closure of the liquid opening of the roller bottle of FIG. 1.

FIG. 2 encompasses bottom planar and side elevation views of the roller bottle of FIG. 1.

FIG. 3 is a cross-section of the roller bottle of FIG. 1 in stacked relationship with a similar bottle including a gas-permeable cap.

### DETAILED DESCRIPTION OF THE INVENTION

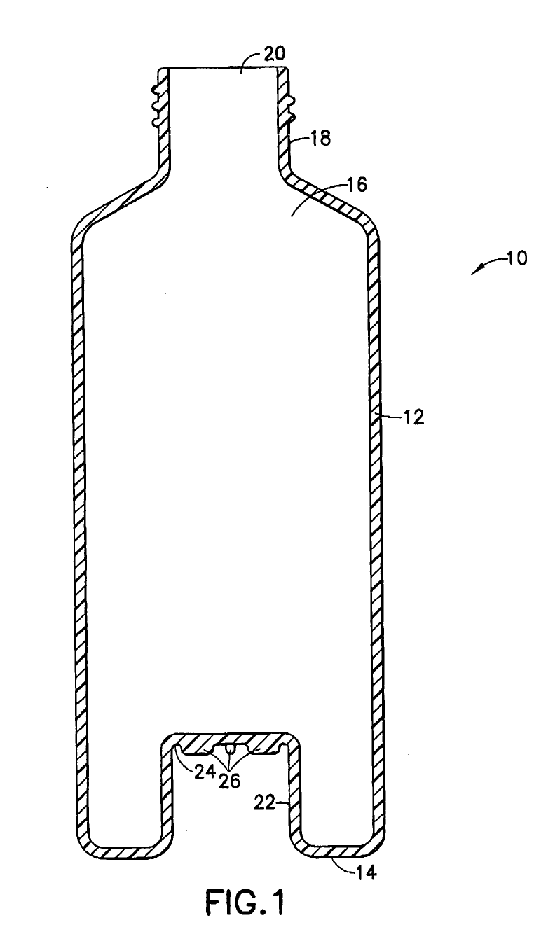

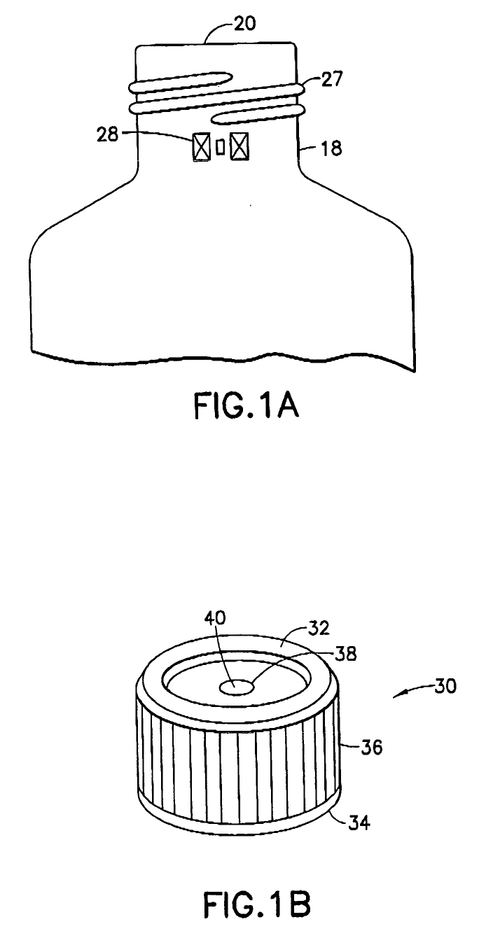

Referring now to the drawings in which like reference characters refer to like parts throughout, FIG. 1 shows a container for cell growth culturing in accordance with the present invention. In particular, FIG. 1 shows roller bottle 10. As can be seen in FIG. 1, roller bottle 10 includes a cylindrical wall 12 which extends from closed bottom end 14 to a top 16. Extending from top 16, and integral therewith, is a projecting neck portion 18 defining a liquid opening 20 at the end opposite the closed end 14. Neck 18 is integral with the bottle 10 and defines a cylindrical conduit having one end integral with the container and the other end defining an opening through which the cells and culture fluids may be introduced into the body of the container. Closed end 14 of bottle 10 includes an inwardly directed recessed portion 22 which is generally frustoconical in shape. The frustoconical portion is shaped and proportioned to correspond to a projecting neck of a similar second bottle so as to accommodate the neck of the second bottle when the two are adjacently stacked end-to-end.

With reference now to FIG. 1A, it can be seen that the neck portion 18 of the roller bottle 10 of FIG. 1 may include external screw threads 27 for receiving an internally screw threaded cap thereon as will be described below. It is anticipated that other cap connections such as bayonette connections may also be used. It is noted that neck 18 may include a locking arrangement 28 for holding a cap in a locked open position on the roller bottle for maintaining the roller bottle open to the environment surrounding it.

As shown in FIG. 1B, a roller bottle according to the present invention may be provided with a cap 30 for elongate neck portion 18. Cap 30 has a top surface 32 and an annular outer skirt 36 extending from the top surface to a bottom stop ledge 34. Cap 30 further has a central orifice 38 extending through with top surface 32. A gas-permeable membrane 40 is affixed to the interior of surface 32 to close orifice 38.

The gas-permeable membrane 40 may be made from any suitable gas-permeable material so long as it provides free passage of gases such as oxygen and carbon dioxide into the hollow chamber defined by the cylindrical wall of the container of the present invention, while preventing bacteria and fungi from passing therethrough. Membrane materials provide adequate rates of carbon dioxide and oxygen permeability while preventing passage of microorganisms. Suitable gas-permeable materials include polyethylene, polycarbonate, acrylic co-polymers and polytetrafluoroethylene.

A problem associated with prior art roller bottles has been the tendency for the planar wall 24 of the recessed portion 22 at the bottom end 14 to seal up against the neck portion 18 of a similar bottle when the two are adjacently stacked. In situations where a vented cap is employed, it is necessary to prevent such a seal so as to allow rapid and uniform equilibration between the gases in the incubator and the bottle, encouraging cell growth within the bottle. In the presence of a seal between the bottom of a bottle and the top of another bottle, gases would not be allowed to flow in and out of the bottle. This may lead to low cell yield, and likely cell death due to the absence of a controlled cell culture environment. For example, as described above, in the absence of an adequate infusion of carbon dioxide into the bottle, the pH of the culture system will not be maintained near desired physiologic levels.

The present invention solves a need in the art by contracting the recessed portion 22 to have a plurality of projecting ribs 26 on a planar surface 24. Ribs 26 are integral with planar surface 24 and extend therefrom so as to allow spacing of the neck portion 18 of a similar bottle from planar wall 24. Ribs 26 prevent the planar surface from sealing against the top of another bottle when the two are adjacently stacked. The ribs may vary in shape, as well as the extent to which they project from planar surface 24. In general, the more the ribs project from the planar surface, the greater the space created between the neck portion of the adjacently stacked similar bottle and the planar wall of the recessed portion of the container of the invention.

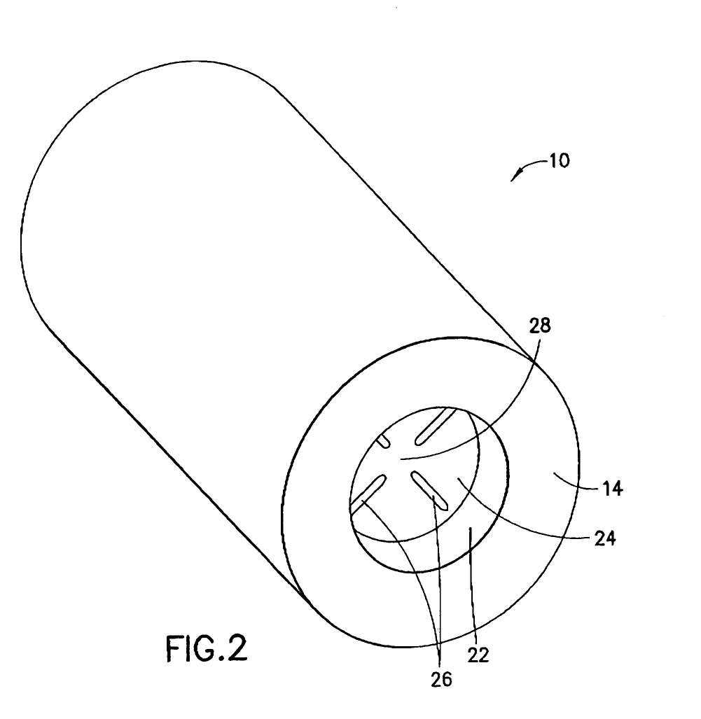

Referring now to FIG. 2, one embodiment is shown wherein the ribs 26 projecting from planar surface 24 extend from a point 28 proximal to the longitudinal axis of the container of the present invention toward recess wall 22 where they terminate, the ribs being about equally spaced about the longitudinal axis of the container.

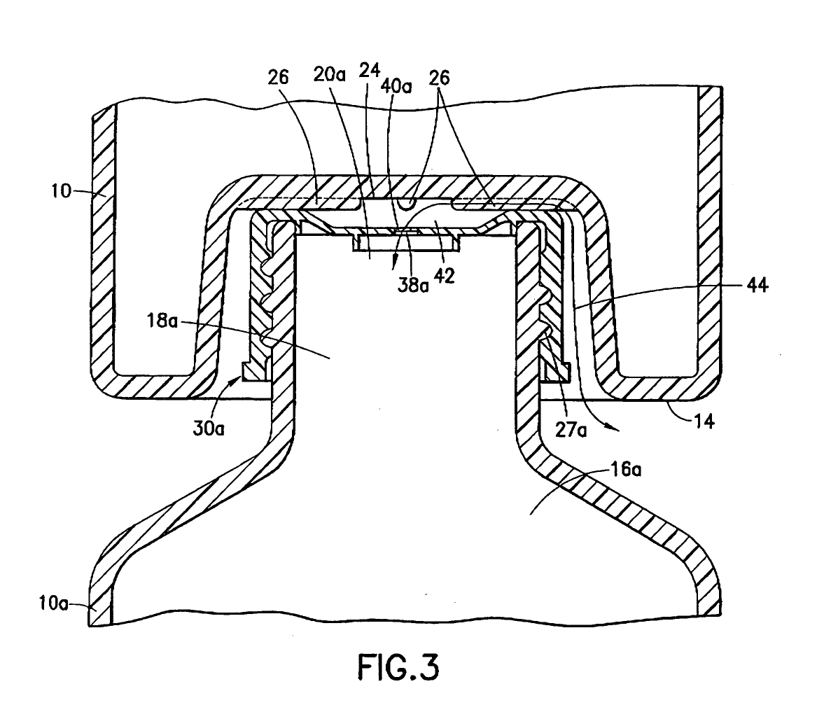

As shown in FIG. 3, when one container 10 is stacked on a similar container 10a, the ribs 26 of bottle 10 extending from planar surface 24 create a space between surface 24 of bottle 10 and neck portion 18a of bottle 10a. In particular, cap 30a of bottle 10a rests against ribs 26 of bottle 10. This allows sufficient space 42 to be created between the top of cap 30a of bottle 10a and surface 24 of bottle 10 to allow gases 44 to flow in and out of orifice 38a of cap 30a. In one embodiment, cap 30a is provided with gas-permeable membrane 40a which allows rapid equilibration of gases between the inside and outside of bottle 10a.

Whereas the container of the present invention is shown in the drawings as having a smooth cylindrical wall, it is well within the contemplation of the present invention that the container may have pleated or corrugated surfaces extending longitudinally or axially about the roller bottle. For example, it is known that pleats formed in the walls of the roller bottle increase the effective surface area for cell growth.

The container of the present invention may be produced as a single one-piece or unitary structure by a simple blow-molding technique. Because of this, the roller bottle of the invention may be mass-produced inexpensively. Moreover, the roller bottle of the invention is configured so that it may be used in a conventional laboratory roller bottle apparatus.

In viewing the conditions for producing roller bottles in accordance with the invention, a variety of thermoplastic materials may be utilized, including, for example, polystyrene, polyethylene terephthalate, the polyolefins and polyvinyl chloride. Polystyrene is particularly desirable because it has been found that cells appear to grow better and in greater numbers on this material.

With the construction of the container of the present invention described herein, stacking with containers of like size and shape is possible. In particular, as shown in FIG. 3, containers such as roller bottles according to the present invention may be arranged adjacently as shown in FIG. 3 so that there is no lost space between adjoining containers, while at the same time eliminating the possibility of forming an air-tight seal between the planar wall of the recess of the bottom of one inventive container and the neck portion of a similar second container.

What is clamed is:

1. A container for cell growth culturing comprising:
an elongate cylindrical wall having a closed bottom end and an opposed projecting neck portion end defining a liquid opening, said closed bottom end including an inwardly directed recessed portion for accommodating a neck portion end of an adjacent stacked similar container, said recessed portion including a planar surface having at least one rib extending therefrom for defining a space between said neck portion of said similar container and said planar surface.

2. The container of claim 1, wherein said space permits gases to enter into and out of a liquid opening of said adjacent stacked similar container.

3. The container of claim 1, wherein said container is a stackable roller bottle.

4. The container of claim 1, wherein said rib is integral with said planar surface.

5. The container of claim 1, wherein said recessed portion further includes a side wall.

6. The container of claim 5, wherein said rib radiates from a point proximal to the longitudinal axis of the container toward said side wall of said recessed portion where said rib terminates.

7. The container of claim 6, wherein a plurality of said ribs is provided, and said ribs are about equally spaced about the longitudinal axis of the container.

8. The container of claim 1, wherein said recessed portion is generally frustoconical in shape.

9. The container of claim 1, wherein said neck includes integral external screw threads for receiving an internally screw threaded cap thereon

10. A roller bottle assembly comprising:
the container of claim 9; and
an internally screw threaded cap, said cap having a top surface and an annular outer skirt extending from said top surface to a bottom stop ledge.

11. The assembly of claim 10, wherein said cap further includes a central orifice extending through said top surface; and a gas permeable membrane which is affixed to an interior side of said top surface to close said orifice.

12. The assembly of claim 10, wherein said recessed portion of said container further includes a side wall.

13. The assembly of claim 12, wherein said rib radiates from a point proximal to the longitudinal axis of the container toward said side wall of said recessed portion where said ribs terminate.

14. The assembly of claim 13, wherein a plurality of said ribs is provided, and said ribs are about equally spaced about the longitudinal axis of the container.

15. A method of stacking containers for cell growth culturing comprising:
providing a first container having a closed bottom end and an opposed projecting neck portion end defining a liquid opening, said closed bottom end including an inwardly directed recessed portion, said recessed portion including a planar surface having at least one rib extending therefrom;
providing a second container; and
stacking said first container and said second container with said neck portion of said second container being nested in said recessed portion of said first container, wherein said rib spaces said neck portion of said second container from said planar surface of said first container.

16. The method of claim 1, wherein said first and said second containers are stackable roller bottles.

17. The method of claim 1, wherein said rib is integral with said planar surface.

18. The method of claim 1, wherein said recessed portion further includes a side wall.

19. The method of claim 18, wherein said rib radiates from a point proximal to the longitudinal axis of the container toward said side wall of said recessed portion where said rib terminates.

20. The method of claim 19, wherein a plurality of said ribs is provided, and said ribs are about equally spaced about the longitudinal axis of the container.
